(19) United States
(12) Patent Application Publication
(10) Pub. No.: US 2008/0262104 A1
(43) Pub. Date: Oct. 23, 2008

(54) Aqueous Polyvinyl Acetate Dispersions Having High Shearing Stability

(75) Inventors: Maximilian Angel, Schifferstadt (DE); Karl Kolter, Limburgerhof (DE)

Correspondence Address: CONNOLLY BOVE LODGE & HUTZ, LLP, P O BOX 2207, WILMINGTON, DE, 19899, US

(73) Assignee: Basf SE, Ludwigshafen (DE)

(21) Appl. No.: 12/065,525

(22) PCT Filed: Aug. 23, 2006
(86) PCT No.: PCT/EP06/65590

§ 371 Date: Mar. 3, 2008

(30) Foreign Application Priority Data
Sep. 2, 2005 (DE) 102005042039.7

(51) Int. Cl. A61K 47/32; C08L 33/02

(52) U.S. Cl. 514/772.6; 525/221

(57) ABSTRACT

Processes comprising: providing a monomer composition comprising a vinyl acetate monomer component and second monomer component comprising an additional monomer selected from the group consisting of acrylic acid, methacrylic acid and mixtures thereof in an amount of 0.05 to 5% by weight, wherein the vinyl acetate monomer component and the second monomer component comprise 100% by weight of the monomer composition; and free-radically polymerizing the monomer composition in the presence of 0.001 to 1% by weight of an ionic emulsifier, based on the weight of the monomer composition, to form an aqueous polymer dispersion; polymer dispersions and powders prepared therewith; and uses of such dispersions and powders in pharmaceuticals and/or cosmetics.

The present invention relates to polyvinyl acetate dispersions with improved shear stability and to a process for preparing such dispersions.

Polyvinyl acetate dispersions and their use for a large number of applications are known per se.

U.S. Pat. No. 5,252,704 discloses polymer powders which are redispersible in water and which are prepared by using polyvinylpyrrolidone (PVP) as dispersing agent. The polymer powders are prepared by employing inter alia vinyl esters in a conventional emulsion polymerization. Before the spray drying, PVP is added to the emulsion. The polymer powders are intended in particular as additives to cement mixtures.

DE 197 09 532 A describes the use of redispersible polymer powders or polymer granules for coating pharmaceutical or agrochemical formulations, where the powders or granules consist of from 10 to 95% by weight of polyvinyl acetate and from 5 to 90% by weight of an N-vinylpyrrolidone-containing polymer and, if appropriate, further additives.

The German patent application P 102004011349.1 describes the preparation of redispersible polymers, in particular of (meth)acrylates, in the presence of ionic emulsifiers. Polyvinyl esters are also mentioned very generally.

Pharmaceutical formulations are often produced as in DE 197 09 532 by employing polymer powders which must be redispersed in water to produce these formulations. The reasons for this production of redispersible powders are that appropriate microbiological stabilization of the aqueous preparations, so that they satisfy the stringent requirements to be met by starting materials for medicaments, is often impossible. Thus, there must be no microbiological attack and also no particle coarsening or even coagulation or sedimentation, because reliable production of the medicament is endangered thereby. The aqueous preparations are often stable only for some weeks. In order to prolong the shelf life, the aqueous preparations are converted into powders from which in turn an aqueous preparation must be produced before use by stirring into water.

The shear stability of such dispersions must therefore satisfy more stringent requirements, especially for pharmaceutical and cosmetic applications in order to prevent coagulation.

WO 02/26845 describes the preparation of polyvinyl acetate dispersions by free-radical polymerization of vinyl acetate in the presence of protective colloids and ionic emulsifiers. However, the dispersions obtained in this way still leave room for improvements in terms of shear stability.

The fact that stabilizers in pharmaceutical dispersions have an adverse effect on film formation has long been known. Thus, it is stated in “Aqueous Polymeric coatings for Pharmaceutical Dosage Forms”, 2nd Edition, Ed. James W. McGinity 1997, Marcel Dekker Inc., New York, pages 56-67, that surfactants and water-soluble additives have very disadvantageous effects on film formation and the properties of films, There is particular mention of sodium lauryl sulfate which alters the structure, the mechanical and permeation properties of films because it migrates into small spaces between the polymer particles during film formation. Hydrophilic compounds such as polyethoxylates may also have very disadvantageous effects on films. Thus, for example, phase separation, flocculation, increased rate of active ingredient release, deterioration in mechanical properties, crystallization of the emulsifiers and similar unwanted phenomena occur.

It was an object of the present invention to find aqueous polyvinyl acetate dispersions having improved shear stability with a minimal emulsifier content.

We have found that this object is achieved by aqueous polyvinyl acetate dispersions which are obtainable by free-radical polymerization of vinyl acetate in the presence of from 0.05 to 5% by weight of acrylic acid or methacrylic acid or mixtures thereof, with the proviso that the total weight of monomers is 100% by weight, and from 0.001 to 1% by weight of at least one ionic emulsifier, based on the total weight of the monomers.

The amount of acrylic acid or methacrylic acid is preferably from 0.05 to 0.9 to . . . % by weight, particularly preferably from 0.1 to 0.6% by weight.

Acrylic acid is preferably employed.

It is possible to provide a conventional anionic emulsifier as ionic emulsifier in the dispersion of the invention, such as, for example, alkali metal and ammonium salts of alkyl sulfates (alkyl radical: C₈ to C₁₆), of alkylsulfonic acids (alkyl radical: C₈ to C₁₆), of sulfuric hemiesters of ethoxylated alkanols (EO degree: 4 to 100, alkyl radical: C₁₂ to C₁₆), and ethoxylated alkyl phenols (EO degree 3 to 50, alkyl radical: C₄ to C₁₂), and of alkylarylsulfonic acids (alkyl radical: C₉ to C₁₈). Further anionic emulsifiers which have proved advantageous are compounds such as Dowfax 2A1 (Dow Chemical Company brand). Sodium lauryl sulfate is preferred. The ionic emulsifier is employed in concentrations of from 0.001 to 1% by weight, preferably from 0.1 to 0.5% by weight, based on the total monomer content.

It is possible if desired additionally to employ conventional nonionic emulsifiers. However, the use of only an anionic emulsifier is preferred.

In a preferred embodiment, the dispersions comprise a protective colloid. The protective colloid is preferably present in an amount of between 1 and 20% by weight, particularly preferably from 5 to 12% by weight, based on the total weight of monomers. The protective colloid present in the dispersion of the invention is preferably polyvinylpyrrolidone, which particularly preferably has a K value of from 20 to 40. It is possible additionally to employ further water-soluble or water-swellable protective colloids such as, for example, cellulose derivatives, preferably hydroxypropylmethylcellulose, methylcellulose or hydroxyethylcellulose, galactomannan, pectin, xanthan, polyvinyl alcohol, acrylate-methacrylate copolymers, sodium carboxymethyl starch, cellulose, degraded starches, maltodextrins etc. The protective colloids may moreover be added before, during and after the polymerization. Addition to the polymerization is preferred.

The present invention also relates to a process for preparing an aqueous polymer dispersion in which vinyl acetate is polymerized by free-radical polymerization in the presence of at least one ionic emulsifier, wherein the polymerization takes place in the presence of from 0.05 to 5% by weight of acrylic acid or methacrylic acid or mixtures thereof, with the proviso that the total weight of monomers is 100% by weight, and from 0.001 to 1% by weight of at least one ionic emulsifier, based on the total amount of monomers.

Accordingly, a mixture of from 95 to 99.95% by weight of vinyl acetate monomer and from 0.05 to 5% by weight of acrylic acid or methacrylic acid monomer, or mixtures of said acids, is polymerized by emulsion polymerization initiated by free radicals.

The polymerization preferably takes place as emulsion polymerization in water in the presence of a protective colloid. In this case, the ratio between protective colloid and ionic emulsifier is at least 4:1 and is up to 100:1 by weight. The ratio between monomers and protective colloid is between 99:1 and 4:1, preferably between 19:1 and 4:1 by weight.

The free-radical initiator employed for the polymerization is preferably Na, K or ammonium peroxodisulfate, but other free-radical initiators which are customary per se, such as hydrogen peroxide, organic peroxides, hydroperoxides or azo compounds—also in conjunction with redox components such as, for example, ascorbic acid—are also possible.

It may moreover be advisable to carry out the polymerization in the presence of a buffer system. The buffer system employed according to the invention is preferably a reagent with a basic action, such as, for example, salts of an acid selected from the group consisting of carbonic acid, boric acid, acetic acid, citric acid and phosphoric acid.

The aqueous polymer dispersion of the invention is preferably a “regulated” polymer dispersion, i.e. the polymerization is carried out in the presence of a polymerization regulator, particularly suitable regulators being sulfur-containing compounds such as, for example, thioglycol, t-dodecyl mercaptan, n-dodecyl mercaptan and ethylhexyl thioglycolate, which lead inter alia to the K values which are preferred according to the invention, of from 45 to 200, particularly preferably 60 to 100, especially 65 to 85, measured on a 0.1% by weight solution in tetrahydrofuran, being settable and to the resulting polymers having sulfur-containing end groups. The total amount of regulator, normally between 0.05 and 1%, preferably between 0.1 and 0.5%, in each case based on the total monomer content, is preferably introduced into the emulsion feed.

The emulsion polymerization is carried out in a manner known per se at temperatures of from 40° C. to 95° C., preferably at temperatures below 80° C.

This process is preferably carried out as a semicontinuous feed process, with the total amount of protective colloid being present in the initial charge. A further preferred procedure entails more than 50 of 100 parts of the ionic emulsifier being present in the initial charge.

The dispersions obtained by the process of the invention normally have a solids content of from 10 to 45% by weight, preferably from 15 to 35% by weight.

The dispersions of the invention are distinguished by high stability with a comparatively low emulsifier content. The process of the invention has the advantage of improved reproducibility in relation to the properties of the resulting dispersions. The further use properties are also extremely satisfactory by comparison with homopolyvinyl acetate dispersions.

Particularly important for producing coatings is—besides the molecular weight and the K value—the particle size of the dispersion particles. The polymer dispersion of the invention therefore preferably has dispersion particles which have an average particle size of only 50 to 300 nm, preferably from 100 to 200 nm. Determination takes place in a conventional way, e.g. by means of an ultracentrifuge, photon correlation spectroscopy or by determination of the transmittance of light. The particle size is normally controlled by the emulsifier concentration. The dispersion particles obtained according to the invention are very fine particles although only a very small amount of an emulsifier is used in the polymerization.

The invention additionally relates to pharmaceutical and cosmetic formulations comprising the polymer of the invention as coating agent and/or film-forming excipient, where the polymer, which can be employed both as aqueous dispersion and as polymer powder, is obtainable by the process described at the outset.

The pharmaceutical formulations are preferably inter alia film-coated tablets, film-coated microtablets, sugar-coated tablets, coated pastilles, capsules, crystals, granules or pellets.

Active pharmaceutical ingredients which can be employed according to the invention besides medicinal substances are also vitamins, dietary supplements or dietetic active ingredients.

The formulations display delayed release of the active ingredient. Such forms are also referred to as forms with sustained release. Sustained means in this connection that the release takes place over a period of from 4 to 36 hours and that the release of 80% of the active ingredient has taken place after four hours at the earliest. Sustained release forms differ in this way from fast-release forms with which 80% of the active ingredient are released after one hour at the latest.

Release from the forms is independent of pH. A pH-independent release means that the release in simulated gastric fluid (0.1 N HCl; pH 1.2) does not differ from the release in simulated intestinal fluid (phosphate buffer; pH 6.8). This means that the difference in release should be not more than 10% at every time of testing.

Dispersions to be used for pharmaceutical formulations may additionally be admixed with conventional pharmaceutically acceptable excipients selected from the group consisting of antifoams, fillers, colorants, pigments, antioxidants, preservatives, gloss improvers and plasticizers in the amounts customary therefor. In the case of active ingredients of low solubility, it may be advisable to add a pore former in addition to control release. Examples of suitable pore formers are hydroxypropylmethylcellulose, polyvinyl alcohol-polyethylene glycol graft copolymers such as Kollicoat® IR, polyvinyl alcohols, vinylpyrrolidone homopolymers and copolymers, natural polysaccharides, or low molecular weight water-soluble substances such as sugars, sugar alcohols, inorganic salts. Such pore formers may depending on the solubility in amounts of from 1 to 30, preferably 2 to 20% by weight based on film former.

The total proportion of excipients may be up to 50% by weight based on the solids content of the dispersion.

A further possibility is also to employ water-insoluble but water-swellable substances of small particle size such as microcrystalline cellulose, crosslinked sodium carboxymethylcellulose or starch and crosslinked polyvinylpyrrolidone.

A particular advantage of the aqueous dispersions is inter alia their low tendency to foaming and thus reduced tendency to pitting. The dispersions additionally display very good stability, as is evident from the shear stability and sedimentation stability. The coatings obtained according to the invention display further advantages besides the low emulsifier content. Thus, the tackiness of the films is reduced in relation to that of comparable commercial compositions. In addition, the coatings display excellent mechanical properties.

The reduced proportion of emulsifier and the improved film formation result in more sustained release and fewer changes in active ingredient release on storage. Curing effects, i.e. changes in the active ingredient release at elevated temperature are likewise minimal or entirely absent.

The dispersions of the invention can also be employed mixed with other dispersions. Thus, for example, they can be employed in combination with acrylate dispersions, for example with the commercially available Kollicoat® or Eudragit® types. These include for example polyacrylate dispersion 30 per cent Ph. Eur. (Kollicoat® EMM 30 D, Eudragit® NE 30 D), ammonio methacrylate copolymer type A and B USP/NF (Eudragit® RL and RS) and methacrylic acid copolymer type C (Kollicoat® MAE 30 DP, Eudragit® L 30-55). Also particularly suitable are the combinations with acrylate dispersions as described in German patent application P 102004011349.1. The dispersions of the invention can also be combined very well with ethylcellulose dispersions such as, for example, Aquacoat® ECD or Surelease®, with an improvement in particular in the flexibility of the ethylcellulose which is brittle per se.

Preparation of the Polymers

General Method

The polymerization vessel was flushed with nitrogen. The deionized water, the sodium laurylsulfate (as 15% by weight aqueous solution) and Kollidon 30, and then part of feed 02 were put into the polymerization vessel (initial charge) while stirring (120 rpm). The initial charge was heated to an internal temperature (IT) of 75° C. At IT 65° C., feed 01 (7% by weight aqueous sodium peroxodisulfate solution) was rapidly added. When 75° C. was reached, feeds 02 (emulsion feed) and 03 (7% by weight aqueous sodium peroxodisulfate solution) were started. Feeds 02 and 03 were added over a period of 2 and 3 hours respectively. After feeds 02 and 03 were complete, polymerization was continued for two hours.

The mixture was then heated to 85° C. and vacuum-distilled for 2 h (under about 200 mbar).

The polymerization mixture was cooled to 20° C. and adjusted to pH 4.5 to 5 with 1% strength sodium hydroxide solution.

Kollidon 30=polyvinylpyrrolidone with K value of 30 (BASF)

### EXAMPLE 1

A dispersion with a VAc/Aa monomer ratio=99.5/0.5% by weight was prepared by the general method. The proportion of emulsifier was 0.25% by weight based on the monomer weight.

Initial Charge

559.0 g of deionized water

4.80 g of sodium laurylsulfate (15% by weight aqueous solution)

40.00 g of Kollidon 30

85.6 g of feed 02

Feed 01

8.7 g of sodium peroxodisulfate (7% by weight aqueous solution)

Feed 02

450.3 g of deionized water

0.28 g of sodium laurylsulfate (solid, i.e. 100% by weight)

2.0 g of sodium acetate×3 H2O

398.0 g of vinyl acetate

2.0 g of acrylic acid

0.96 g of t-dodecyl mercaptan

Feed 03

17.6 g of sodium peroxodisulfate (2.5% by weight aqueous solution)

The bluish white aqueous dispersion had a solids content of 29% by weight. The average particle size was 160 nm. The K value, measured in a 0.1% by weight solution in THF, was 75.

### EXAMPLE 2

The procedure was as in example 1. However, 6.35 g of sodium laurylsulfate (as 15% by weight solution) were used in the initial charge and 0.37 g of sodium lauryisulfate (solid, i.e. 100% by weight) was used in feed 02. 2-Ethylhexyl thioglycolate was used instead of t-dodecyl mercaptan.

The proportion of emulsifier was 0.33% by weight based on the monomer weight.

The bluish white aqueous dispersion had a solids content of 29% by weight. The average particle size was 150 nm. The K value, measured in a 0.1% by weight solution in THF, was 73.

COMPARATIVE EXAMPLE A

The polymerization was carried out using 100% by weight of vinyl acetate as monomer in analogy to the preparation in example 1. No stable dispersion resulted.

Determination of the Shear Stability

General Method

The dispersion to be tested is passed through a sieve with a mesh width of 125 μm, and 100 g of the filtrate are weighed into a 250 ml glass beaker. A studded stirrer (d=36 mm with 4 studs of 5×3 mm on the Outer ring and, at a distance of 5 mm, 4 studs of 4×3 mm on the inner ring, all 1 cm long) is clamped in the stirrer motor. The under edge of the studded stirrer should be at a distance of 0.5 cm from the base of the glass beaker. The glass beaker is likewise fastened and the dispersion is stirred at 2000 rpm for 15 min and subsequently poured through a sieve with a mesh width of 125 μm. Any resulting residue is transferred to a filter which has previously been dried at 105° C. for one hour and then weighed.

The filter with the residue is then dried again in a drying oven at 105° C. for one hour.

After cooling in a desiccator, the filter is reweighed on an analytical balance.

For the evaluation, the amount coagulated on stirring is calculated:

$$Amount coagulated on stirring in \% = \frac{( filter + residue - weight of empty filter ) \times 100}{solids content in the dispersion in \%}$$

A larger amount coagulated on stirring means a lower shear stability of the tested dispersion.

### COMPARATIVE EXAMPLE B

For comparison, the shear stability of a pure polyvinyl acetate dispersion with an emulsifier content of 1.004% by weight according to example 1 of WO 02/26845 was determined.

| Dispersion of | % by weight coagulated on |
|---|---|
| Ex. No. | stirring |
| 1 | 0 |
| 2 | 0 |
| A | 3 |
| B | 0.15 |

1.-16. (canceled)

17. An aqueous polymer dispersion prepared by a process comprising:
providing a monomer composition comprising a vinyl acetate monomer component and second monomer component comprising an additional monomer selected from the group consisting of acrylic acid, methacrylic acid and mixtures thereof in an amount of 0.05 to 5% by weight, wherein the vinyl acetate monomer component and the second monomer component comprise 100% by weight of the monomer composition; and
free-radically polymerizing the monomer composition in the presence of 0.001 to 1% by weight of an ionic emulsifier, based on the weight of the monomer composition.

18. The aqueous polymer dispersion according to claim 17, wherein the second monomer component is present in an amount of 0.1 to 0.6% by weight.

19. The aqueous polymer dispersion according to claim 17, wherein the second monomer component comprises acrylic acid.

20. The aqueous polymer dispersion according to claim 17, wherein the ionic emulsifier comprises an anionic emulsifier.

21. The aqueous polymer dispersion according to claim 17, wherein the ionic emulsifier is an anionic emulsifier.

22. The aqueous polymer dispersion according to claim 17, wherein the ionic emulsifier comprises sodium lauryl sulfate.

23. The aqueous polymer dispersion according to claim 17, further comprising a protective colloid.

24. The aqueous polymer dispersion according to claim 23, wherein the protective colloid comprises a polyvinylpyrrolidone having a K value of from 20 to 40.

25. The aqueous polymer dispersion according to claim 17, wherein the ionic emulsifier is present in an amount of 0.1 to 0.5% by weight.

26. The aqueous polymer dispersion according to claim 17, wherein the dispersion comprises particles having an average particle size of 50 to 300 nm.

27. The aqueous polymer dispersion according to claim 17, wherein the dispersion comprises particles having an average particle size of 100 to 200 nm.

28. A process comprising:
providing a monomer composition comprising a vinyl acetate monomer component and second monomer component comprising an additional monomer selected from the group consisting of acrylic acid, methacrylic acid and mixtures thereof in an amount of 0.05 to 5% by weight, wherein the vinyl acetate monomer component and the second monomer component comprise 100% by weight of the monomer composition; and
free-radically polymerizing the monomer composition in the presence of 0.001 to 1% by weight of an ionic emulsifier, based on the weight of the monomer composition, to form an aqueous polymer dispersion.

29. The process according to claim 28, wherein the polymerization takes place in the presence of a protective colloid.

30. A polymer in powder form prepared by a process comprising drying an aqueous polymer dispersion according to claim 17.

31. A pharmaceutical or cosmetic formulation comprising an aqueous polymer dispersion according to claim 17.

32. A pharmaceutical or cosmetic formulation comprising a powdered polymer according to claim 30.

33. The pharmaceutical or cosmetic formulation according to claim 31, further comprising one or more additional acceptable excipients selected from the group consisting of antifoams, fillers, colorants, pigments, antioxidants, preservatives, pore formers, gloss improvers, plasticizers, and combinations thereof.

34. The pharmaceutical or cosmetic formulation according to claim 32, further comprising one or more additional acceptable excipients selected from the group consisting of antifoams, fillers, colorants, pigments, antioxidants, preservatives, pore formers, gloss improvers, plasticizers, and combinations thereof.

35. A delayed-release pharmaceutical formulation comprising at least one active pharmaceutical ingredient coated with a film prepared from an aqueous polymer dispersion according to claim 17.

36. A delayed-release pharmaceutical formulation comprising at least one active pharmaceutical ingredient coated with a film prepared from a powdered polymer according to claim 30.
